(19) United States
(12) Patent Application Publication
(10) Pub. No.: US 2018/0267019 A1
(43) Pub. Date: Sep. 20, 2018

(54) USE OF PEPTIDE HYDROGEL SCAFFOLDS FOR THREE-DIMENSIONAL THROUGHPUT DRUG DISCOVERY

(71) Applicants: UNIVERSITY OF DELAWARE, Newark, DE (US); THE NEMOURS FOUNDATION, Jacksonville, FL (US)

(72) Inventors: Sigrid A. LANGHANS, Chadds Ford, PA (US); Darrin J. POCHAN, Landenberg, PA (US); Peter WORTHINGTON, Haddon Heights, NJ (US); Andrew D. NAPPER, Dublin, CA (US)

(73) Assignees: UNIVERSITY OF DELAWARE, Newark, DE (US); THE NEMOURS FOUNDATION, Jacksonville, FL (US)

(21) Appl. No.: 15/762,228

(22) PCT Filed: Sep. 23, 2016
(86) PCT No.: PCT/US2016/053393

§ 371 Date: Mar. 22, 2018

Related U.S. Application Data
(60) Provisional application No. 62/232,598, filed on Sep. 25, 2015.

(51) Int. Cl. G01N 33/50; C12Q 1/02

(52) U.S. Cl. CPC G01N 33/502; G01N 33/5044; C12Q 1/025

(57) ABSTRACT

An assay mixture includes a hydrogel of a shear-thinning β-hairpin peptide, a plurality of cells, and one or more predetermined compounds being investigated for ability to affect the growth, viability, reproduction characteristics, or activity of the cells. A high throughput screening device includes a plurality of sample wells adapted for high throughput screening, wherein each well contains the assay mixture. A method of using the high throughput screening device includes a) depositing in each of the wells a β-hairpin hydrogel including the cells; depositing in at least some of the wells one or more of the compounds, either along with the β-hairpin hydrogel or separately; and c) measuring the growth, viability, reproduction characteristics, or activity of the cells in each of the plurality of wells.

### CROSS REFERENCE TO RELATED APPLICATIONS

This application claims priority benefit of U.S. provisional Patent Application. No. 62/232,598, filed 25 Sep. 2015, the entirety of which is incorporated herein by reference for all purposes.

### BACKGROUND OF THE INVENTION

High-throughput screening (HTS) of compound libraries remains a promising initial step in building new classes of lead compounds. However, and particularly in cell line models for cancer, its value is limited in predicting clinical effectiveness. One of the reasons for this lack of reliability to predict in vivo efficacy has often been ascribed to the fact that most HTS screenings were done using traditional 2D cultures of cancer cells where the non-physiological 2D conditions differ from cells grown in the more in vivo like 3D systems. For example, human medulloblastoma cells grown in 3D cultures express is increasingly immature features found in tumors and vary in drug response when compared to cells grown in 2D systems.

Thus, a 3D culture model is expected to be a better platform for drug discovery in cancer and is likely more predictive of efficacy of potential drugs for future preclinical studies and clinical trials. However, while commonly used natural 3D matrices such as collagen or MATRIGEL® matrix provide an in vivo like environment, the capabilities to modify chemical and mechanical properties are limited. Thus, new matrices allowing greater control of these properties would be a welcome addition to the field of drug discovery.

### SUMMARY OF THE INVENTION

In some aspects, the invention provides an assay mixture that includes a hydrogel of a shear-thinning β-hairpin peptide, a plurality of cells, and one or more predetermined compounds being investigated for ability to affect the growth, viability, reproduction characteristics, or activity of the cells.

In some aspects, the invention provides a high throughput screening device that includes a plurality of sample wells adapted for high throughput screening, wherein each well contains the assay mixture. The one or more compounds and/or the amounts thereof may be the same or different from well to well, provided that some but not all of the wells may optionally be control wells containing no compounds to be investigated.

In some aspects, the invention provides a method of using the high throughput screening device for high throughput screening of compounds for ability to affect the growth, viability, reproduction characteristics, or activity of cells. The method includes

a) depositing in each of the wells a β-hairpin hydrogel including the cells;

b) depositing in at least some of the wells one or more of the compounds, either along with the β-hairpin hydrogel or separately; and

c) measuring the growth, viability, reproduction characteristics, or activity of the cells in each of the plurality of wells.

### BRIEF DESCRIPTION OF THE DRAWINGS

FIG. 1 shows encapsulation of medulloblastoma cells in MAX8, collagen, and MATRIGEL® matrix, compared to cells on glass. Shown are z-stack images along the z axis indicating cell location in each hydrogel visualized with syto 13. The images are 250 μm in height. The arrows show the location of the glass cover slip serving as the physical bottom of the sample.

FIG. 2A shows cell viability of medulloblastoma cells (10,000 cells/well in a 96-well plate) encapsulated in 0.25 wt % or 0.5 wt % MAX8, determined using the RealTime-Glo MT Cell Viability Assay at indicated timepoints. Bars represent SD of the mean, n=3. CPS, counts per second.

FIG. 2B shows cell viability of primary mouse cerebellar granule precursor cells from C57BL/6 mice (CGP; 50,000 cells/well seeded in a 96-well plate) encapsulated in 0.5 wt % MAX8. Signals were measured 48 hours after cell encapsulation, and were compared to the baseline signal obtained after cells were allowed to equilibrate for 24 hours after isolation. Data represent mean from five determinations.

FIG. 2C shows cell viability of medulloblastoma cells encapsulated in 0.5 wt % MAX8 or MAX8 functionalized with RGDS, IKVAV or YIGSR sequences. Cell viability was determined at indicated time points using the RealTime-Glo assay. For comparison, non-proliferation cells maintained in serum-free conditions are shown. Bars represent SD of the mean, n=3. CPS, counts per second.

FIG. 2D shows an oscillatory frequency sweep of MAX8-RGDS, indicating a stiff hydrogel. Black lines, G′ (storage modulus); grey lines, G″.

FIG. 2E shows an oscillatory time sweep, showing gelation kinetics before and after shear thinning (dashed line) with immediate rehealing of MAX8-RGDS after network disruption.

FIG. 3A compares the relative quantity of nestin, snail and gli3 in medulloblastoma cells cultured in 2D monolayers and in 3D hydrogel constructs, native or tagged with the indicated adhesive peptides, as measured by qRT-PCR. Bars represent SD of the mean, n=3.

FIG. 3B compares the cell viability of medulloblastoma cells cultured in 3D MAX8-RGDS constructs (left panel) and in 2D monolayers (right panel) treated with vismodegib. Bars represent SD of the mean, n=3.

FIG. 4A shows the cell viability signal measured from an increasing number of cells encapsulated in 0.5wt % MAX8 using the RealTime-Glo assay. N=5; CPS, counts per second.

FIG. 4B shows the cell viability signal measured from an increasing number of cells encapsulated in 0.5wt % MAX8 using the CellTiter-Glo and CellTiter-Glo 3D assays. N=5; CPS, counts per second.

FIG. 4C shows cell growth of medulloblastoma cells encapsulated in 0.5 wt % MAX8 tagged with the RGDS sequence.

FIG. 4D shows viability of untreated control cells, cells treated with ethanol to induce cell death (dead cells), and wells without cells (no cells). Z factor, 0.576; Signal to noise, 9.5; CPS, counts per second.

FIG. 4E shows DMSO tolerance of medulloblastoma cells in MAX8 using a 384-well plate setup.

FIG. 5A is a 3D confocal microscope image showing a live—dead assay of MG63 cells encapsulated in 0.5 wt % MAX8 hydrogel. This image was taken three hours after this hydrogel-cell construct was shear-thin delivered via an 18-gauge syringe needle.

FIG. 5B shows the one-dimensional flow velocity of living MG63 cells through a 250 um-ID capillary at 4.00 mL/h. Solid symbols, aqueous buffer (25mM Hepes, pH 7.4; open symbols, cells encapsulated in 0.75 wt % MAX 8 hydrogel in 25 mM Hepes, pH 7.4. Note the central wide plug flow region where hydrogel material and cell payloads experience little, if no, shear (open circles) in contrast to the laminar flow in buffer (solid circles).

FIG. 5C shows the oscillatory rheology of pure peptide hydrogel (solid symbols) and a hydrogel-drug construct using the chemotherapeutic vincristine as an example (open symbols). Note that the pure peptide and the hydrogel-drug construct exhibit identical mechanical properties. G′ is the storage modulus (triangular symbols), or stiffness, measure in Pascal, and G″ is the loss modulus (square symbols).

FIG. 5D is a display of solid hydrogel properties when in contact with excess aqueous buffer solution.

### DETAILED DESCRIPTION OF THE INVENTION

All references mentioned in the present patent application are incorporated herein by reference for all purposes.

The inventors now disclose assay mixtures providing greater control of the chemical and mechanical properties of matrices for drug discovery, and HTS assay methods using these matrices. These matrices can be optimized to mimic the native extracellular matrix by porosity, permeability and mechanical stability and can provide a biologically active environment for cells to proliferate and differentiate.

The invention provides assay mixtures that comprise a shear-thinning hydrogel of a β-hairpin peptide, a plurality of cells, and one or more predetermined compounds being investigated for ability to affect the growth, viability, reproduction characteristics, or activity of the cells.

U.S. Pat. No. 7,884,185 describes β-hairpin peptides suitable for use according to the invention, and also describes suitable hydrogels using these peptides. In some embodiments, the beta-hairpin peptide is MAX8, as described in that patent. However, any other β-hairpin peptide may be used, non-limiting examples of which include the specific compounds disclosed in that patent and/or in any of the references incorporated herein by reference. Derivatives of MAX8 may also be used, for example MAX8 that has been modified to add a RGD peptide sequence. Hydrogels of MAX8 or other β-hairpin peptides produce matrices that:
[1] are well-defined materials with controllable, desired material properties (stiffness, porosity, nanofibrillar morphology),
[2] display a unique solution assembly that doesn't require covalent crosslinking reactions,
[3] are an injectable solid - once formed with desired solid properties, the material can flow under shear (e.g., when injected) but immediately reheal into a solid hydrogel with the same solid properties prior to shear,
[4] can be handled and automatically dispensed at room temperature,
[5] can immediately assemble into a defined solid hydrogel at physiological conditions,
[6] can encapsulate any desired molecular therapeutic or cells without affecting the hydrogel properties of the material.

The versatility of β-hairpin hydrogels with tunable porosity, permeability and stability, and the possible functionalization with additional moieties (e.g., RGDS peptides, proteolytic sites) to enhance nutrient exchange and cell adhesion and improve cell proliferation makes this material uniquely suited for assaying drugs with a variety of cell types, providing broad applicability. A variety of cell types (mammalian) have been studied to date, and all have been viable in the hydrogels.

Self-assembling and hydrogelating β-hairpin peptides possess all the features of an ideal candidate for development as a versatile 3D cell culture matrix that can be dispensed automatically using standard HTS equipment employing a plurality of wells. The MAX8 (VKVKVKVK-(V^{D}PPT)-KVEVKVKV-NH₂) peptide and its derivatives undergo assembly at physiological conditions into a hydrogel with a well-defined, nanofibrillar matrix, desired porosity and stiffness and can be shear-thin injected as a solid material. Useful derivatives of MAX8 include:

| | |
|---|---|
| MAX8-RGDS | RGDSVKVKVKVK-(V^{D}PPT)-KVEVKVKV-NH₂ |
| MAX8-IKVAV | IKVAVVKVKVKVK-(V^{D}PPT)-KVEVKVKV-NH₂ |
| MAX8-YIGSR | YIGSRVKVKVKVK-(V^{D}PPT)-KVEVKVKV-NH₂ |

The MAX1 (VKVKVKVK-(V^{D}PPT)-KVKVKVKV-NH₂) peptide and its derivatives may also be useful.

Hydrogel properties such as stiffness, network structure and porosity can be modified by using different β-hairpin peptide primary sequences. See Giano, M. C., D. J. Pochan, and J. P. Schneider. 2011. Controlled biodegradation of self-assembling beta-hairpin peptide hydrogels by proteolysis with matrix metalloproteinase-13. Biomaterials. 32:6471-6477; Haines-Butterick, L., K. Rajagopal, M. Branco, D. Salick, R. Rughani, M. Pilarz, M. S. Lamm, D. J. Pochan, and J. P. Schneider. 2007. Controlling hydrogelation kinetics by peptide design for three-dimensional encapsulation and injectable delivery of cells. Proceedings of the National Academy of Sciences of the United States of America. 104:7791-7796; Nagarkar, R. P., R. A. Hule, D. J. Pochan, and J. P. Schneider. 2008. De novo design of strand-swapped beta-hairpin hydrogels. Journal of the American Chemical Society. 130:4466-4474; Nagy, K. J., M. C. Giano, A. Jin, D. J. Pochan, and J. P. Schneider. 2011. Enhanced Mechanical Rigidity of Hydrogels Formed from Enantiomeric Peptide Assemblies. Journal of the American Chemical Society. 133:14975-14977; and Pochan, D. J., J. P. Schneider, J. Kretsinger, B. Ozbas, K. Rajagopal, and L. Haines. 2003. Thermally reversible hydrogels via intramolecular folding and consequent self-assembly of a de Novo designed peptide. Journal of the American Chemical Society. 125:11802-11803).

Hydrogel properties can also be modified by using different solution conditions, such as varying pH (Schneider, J. P., D. J. Pochan, B. Ozbas, K. Rajagopal, L. Pakstis, and J. Kretsinger. 2002. Responsive hydrogels from the intramolecular folding and self-assembly of a designed peptide. Journal of the American Chemical Society. 124:15030-15037) and salt concentration (Ozbas, B., J. Kretsinger, K. Rajagopal, J. P. Schneider, and D. J. Pochan. 2004. Salt-triggered peptide folding and consequent self-assembly into hydrogels with tunable modulus. Macromolecules. 37:7331-7337), including those found under physiological conditions (Branco, M. C., D. J. Pochan, N. J. Wagner, and J. P. Schneider. 2009. Macromolecular diffusion and release from self-assembled beta-hairpin peptide hydrogels. Biomaterials. 30:1339-1347; Yan, C., A. Altunbas, T. Yucel, R. P. Nagarkar, J. P. Schneider, and D. J. Pochan. 2010. Injectable solid hydrogel: mechanism of shear-thinning and immediate recovery of injectable [small beta]-hairpin peptide hydrogels. Soft Matter. 6:5143-5156). Thus physical gel properties can be easily adjusted for different cell lines by modulating peptide sequence, peptide concentration, or ionic strength of the culture medium.

The mechanism of stiff hydrogel formation, shear-thinning for simple injection, and immediate solidification of the β-hairpin peptide hydrogels is well understood and has been demonstrated in physiologically relevant conditions. Due to the fast gelation kinetics under physiological conditions, living cells can be homogenously encapsulated in MAX8 or other β-hairpin peptide hydrogels.

In some embodiments, the stiffness of the assay mixture is within 5%, 10%, 20% or 50% above or below the stiffness of an in vivo tissue in which the growth, viability, reproduction characteristics, or activity of like cells is sought to be affected. In other words, the stiffness of the assay mixture is designed to match the stiffness of the living environment in which the cell would normally be found. For example, the stiffness would match that of brain tissue if the cell is a brain cancer cell.

The ability to encapsulate and shear-deliver in plug flow fashion with cells experiencing minimal injection shear forces allows cells of diverse origin, including primary cells, to be delivered without affecting cell viability.

The MAX8 hydrogel, to take one example, is cyto-compatible with diverse cell lines including mesenchymal stem cells, osteosarcoma, pancreatic cancer and medulloblastoma cells. MAX8 hydrogel-cell constructs retain the same homogeneous cell distribution/microstructure after shear-thin injection as existed prior to injection.

Biological functionalization of MAX β-hairpin hydrogels has been demonstrated, e.g., MMP cleavage site addition for specific degradation mechanism (Giano, M. C., D. J. Pochan, and J. P. Schneider. 2011. Controlled biodegradation of self-assembling beta-hairpin peptide hydrogels by proteolysis with matrix metalloproteinase-13. Biomaterials. 32:6471-6477) or inclusion of an RGD sequence to improve adhesion (Rajagopal, K. 2007. Rational peptide design for functional materials via molecular self-assembly. Ph.D. thesis, Dept. of Chemistry and Biochemistry. University of Delaware, Newark) to provide for a cell-responsive hydrogel construct. These and other modifications to MAX8 are suitable for making assay mixtures according to the invention, provided that the modified MAX8 is still capable of producing a shear-thinning hydrogel.

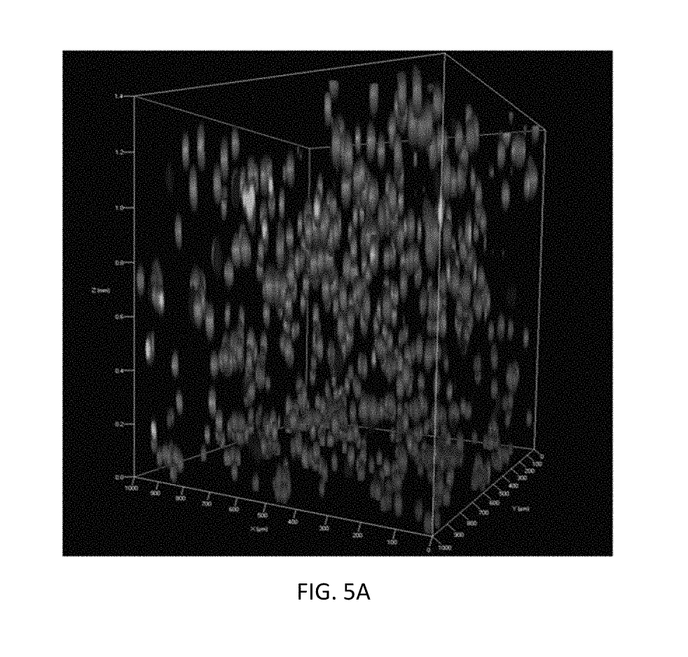

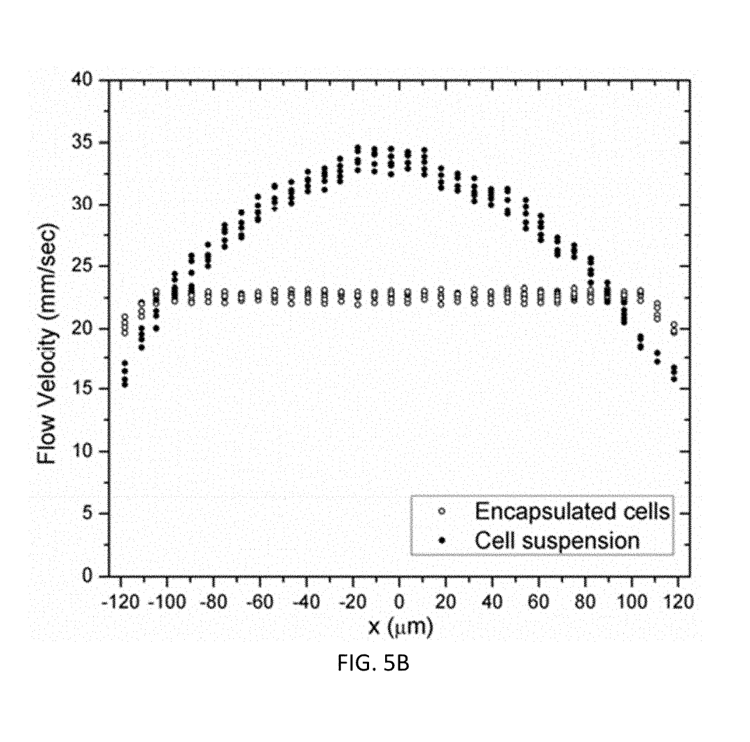

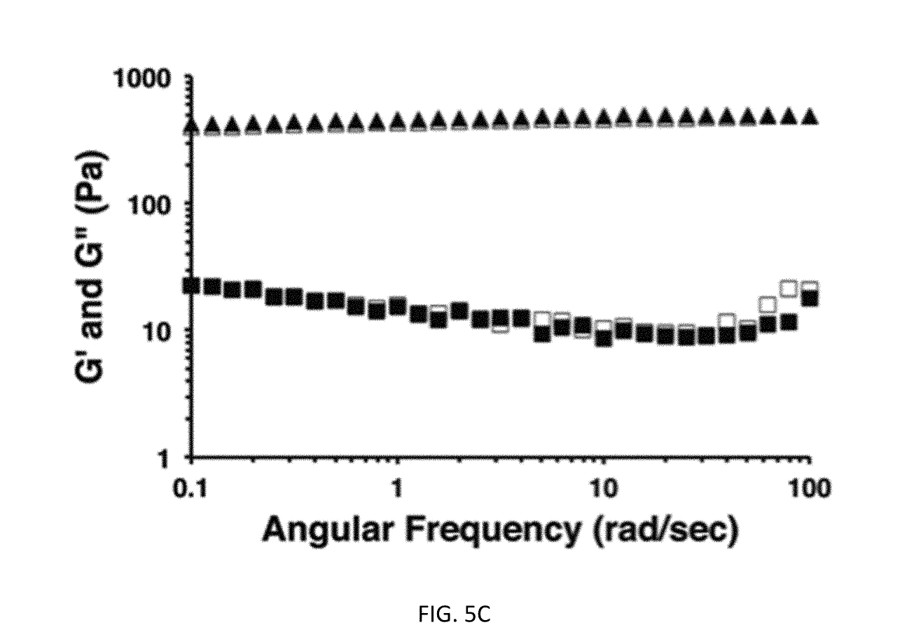

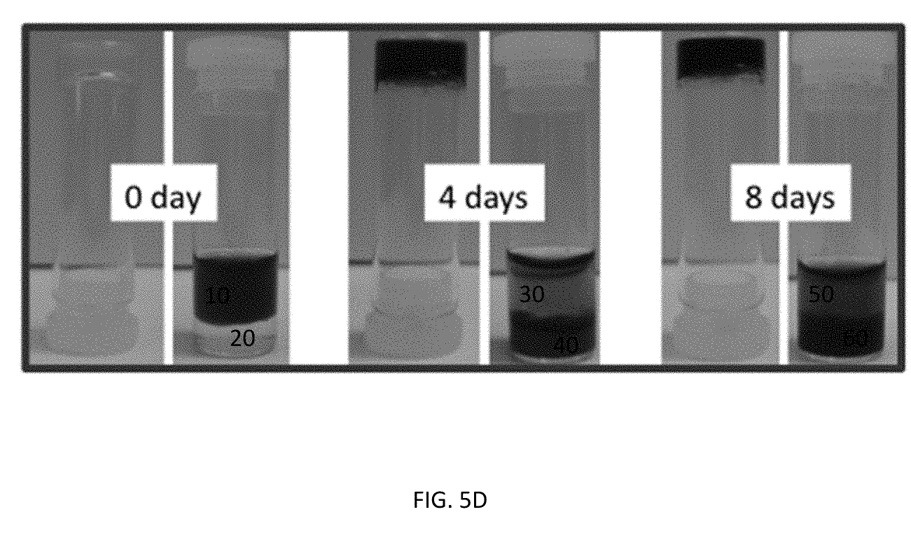

Solid hydrogel-drug constructs of MAX8 exhibit the same material properties as hydrogels without drugs as tested for a wide range of chemical compounds. This allows evaluation of a wide variety of compounds with diverse chemical structures without affecting the intrinsic properties of the 3D culture matrix. These effects are depicted in FIGS. 5A through 5D, using MAX8 hydrogels.

The in vitro drug release of β-hairpin hydrogel-encapsulated compounds, for example small molecules for chemotherapy, is primarily due to slow diffusion. Alternatively, the inventors have found that compounds in buffer solution layered on top of the hydrogel diffuse throughout the hydrogel with the hydrogel remaining intact as a stiff solid without swelling or dissolving.

FIG. 5D is a display of solid hydrogel properties when in contact with excess aqueous buffer solution. Inverted and uprights vials are shown at the left and right, respectively, in each of three panels corresponding to 0, 4, and 8 days elapsed time. At 0 days, the solid hydrogel has been formed with appropriate physiological buffer conditions (inverted vial) and excess buffer solution with blue dye 10 was placed on top of clear hydrogel 20. At 4 days, the blue dye has diffused throughout previously clear hydrogel (inverted vial) and then excess buffer solution with yellow dye 30 was placed on top of blue hydrogel 40. At 8 days the yellow dye has now completely diffused into the blue solid hydrogel, making the hydrogel green 60 (inverted vial) and red buffer solution 50 was placed on top. The hydrogel remains a porous solid with defined properties and does not swell during assays. FIG. 5D demonstrates that, even though the hydrogel is a physical network with no covalent crosslinking, the material behaves as a permanent network with constant volume that maintains material properties during an assay or experiment.

Additionally, because the of β-hairpin hydrogels are deposited as solids, delivery of different cell types, mixtures of cell types, and/or different drugs can be layered in the vials, thus providing a powerful tool for studying complex interactions among the cells and drugs. One or more drugs can be included in the hydrogel prior to injection, coinjected with the hydrogel into the HTS wells, and/or added to the wells either before or after the hydrogels.

Collagen or MATRIGEL® matrix are commonly used 3D matrices that provide an in vivo like environment. However, due to their natural origin the variation in different preparations is considered a major hindrance to obtain reproducible results. Natural matrices also limit the possibility of mimicking different tissue environments as they only have limited capabilities for their chemical and mechanical properties to be modified. MAX8 and other β-hairpin hydrogels can overcome these limitations and provide a versatile HTS-compatible 3D matrix that, unlike collagen or MATRIGEL® matrix, can be handled at ambient temperatures.

One of the limitations of synthetic matrices, including MAX8, is often the lack of adhesive properties. However, inclusion of the RGD peptide sequence into MAX8 is feasible and produces a hydrogel peptide with similar mechanical properties as MAX8 while at the same time increasing cell compatibility. Addition of growth factors encapsulated into the hydrogel can increase growth in hydrogel encapsulated cell cultures. Drug encapsulation, including encapsulation of neurotrophic peptides such as NGF and BDNF, does not affect MAX8 gelation kinetics.

Any of a variety of cell types can be used in assay mixtures according to the invention, with nonlimiting examples being eukaryotic cells, cancer cells, medulloblastoma cells, bacterial cells, fungal cells or spores thereof, and plant cells.

Cells may be distributed randomly or evenly throughout the assay mixture. For example, medulloblastoma cells can be mixed homogeneously throughout entire hydrogel. Cell superstructures (spheroids), for example spheroids formed from medulloblastoma cells, can be encapsulated into the hydrogel.

The hydrogel may be layered. For example, there may be a bottom layer of hydrogel without cells, a middle layer of hydrogel containing one type of cell (e.g., fibroblast cells (3T3)), a top layer of hydrogel without cells, and a cell monolayer (e.g., keratinocytes, human embryonic kidney cells, neurons) cultured on top of the top hydrogel layer.

In some cases, the cells are distributed in a layer of the assay mixture while another layer of the assay mixture contains no cells. The layering may be achieved by sequential deposition of different compositions, at least one of which contains the cells.

In some embodiments, each of the one or more predetermined compounds is distributed randomly or evenly throughout the assay mixture. In some cases, at least one of the one or more predetermined compounds is distributed in a layer of the assay mixture while another layer of the assay mixture contains none of said predetermined compounds.

The hydrogel weight percent can be different for each layer, controlling drug diffusion. Drugs or growth factors can be added in any hydrogel layer while plating the cell culture, or added to the cell culture media at any point following the start of incubation. The hydrogel scaffold can be synthesized to include pertinent cell ligands covalently bonded to the shear-thinning β-hairpin peptide. Non-limiting examples of suitable ligands include RGDS-fibronectin, IKVAV-laminin, YIGSR-laminin, and GFOGER-collagen. Multiple cell types can be co-cultured.

The invention also provides a device comprising a plurality of sample wells adapted for high throughput screening (HTS), wherein each well contains an assay mixture according to the invention, and wherein the one or more compounds and/or the amounts thereof may be the same or different from well to well, provided that some but not all of the wells may optionally be control wells containing no compounds to be investigated.

The invention also provides a method of using the device described above, comprising

a) depositing in each of the wells a β-hairpin hydrogel comprising the cells;

b) depositing in at least some of the wells one or more of the compounds, either along with the β-hairpin hydrogel or separately; and

c) measuring the growth, viability, reproduction characteristics, or activity of the cells in each of the plurality of wells.

Compounds to be evaluated by the high throughput screening are added as part of a hydrogel, either in the hydrogel containing the cells or in a separate hydrogel. Alternatively, the compounds may be added by depositing a non-hydrogel mixture containing them.

### EXAMPLES

MAX8 β-Hairpin Peptide Synthesis

The synthesis and purification of MAX8 β-hairpin peptide has been described previously in detail (Haines-Butterick et al., 2007b; Yan et al., 2012). Synthesis of MAX8 used in the current study was performed with an automated AAPPTEC peptide synthesizer, using standard Fmoc-based solid phase peptide synthesis. For functionalized peptides, the RGDS, IKVAV or YIGSR were added on to the native MAX8 peptide sequence VKVKVKVK-(V^{D}PPT)-KVEVKVKV-NH₂.

Oscillatory Rheology

Rheology measurements were performed on an AR G2 rheometer (TA instruments) with a 20 mm stainless steel parallel plate geometry. After mixing the peptide solution with the buffer solution, the samples (170 μL) were loaded immediately onto the temperature control (37° C.) Peltier plate and mineral oil was added around the circumference of the geometry to prevent dehydration of the hydrogel. Dynamic time sweep experiments (DTS) were performed to monitor the storage (G′) and loss (G″) modulus as a function of time (6 rad/s frequency, 0.2% strain) for 60 min. For shear-thinning experiments, the samples were subjected to 500 s⁻¹ steady-state shear for 60 s after which oscillatory measurement was performed at 6 rad/s frequency, 0.2% strain. Subsequently, recovery of the storage (G′) and loss (G″) modulus as a function of time was monitored for 30 min. Dynamic frequency (0.1-100 rad/s frequency, 1.0% strain) sweep experiments were performed to establish the frequency response of the samples. All measurements were performed in triplicates.

Preparation of Basic Hydrogel-Cell Constructs

Human medulloblastoma cells were propagated in Dulbecco's Minimum Essential Media (DMEM) supplemented with 10% fetal bovine serum and penicillin-streptomycin-glutamine at 37° C. and 5% CO₂ using standard 2D cell culture protocols and tissue-culture treated plastic cell ware. For isolation of primary cerebellar granule precursor (CGP) cells, the cerebellum was dissected from P4-6 pups of C57BL/6 mice and dissociated into single cells using the Papain Dissociation System kit (Worthington Biochemical Corp, Freehold, N.J.). After filtering through a nylon mesh (70 μm pore size), the cells were briefly centrifuged and resuspended in Neurobasal medium supplemented with 0.25 mM KCI and B27.

In general and unless otherwise indicated, MAX8-cell constructs were prepared as 0.25 wt % MAX8 hydrogels. First, the peptide was dissolved in 50 mM HEPES buffer (pH 7.4) (0.25 μg MAX8 per 100 μL of hydrogel) and then an equal volume of single cell suspension in DMEM was added and gently mixed. Mixing the MAX8 solution with the culture medium triggers the intramolecular folding of the peptide, resulting in self-assembly into a hydrogel. For additional hydrogels with different concentrations of MAX8, the amount of peptide was adjusted but otherwise the same hydrogel assembly protocol was followed. The same procedures were followed when functionalized MAX8 was used to prepare hydrogel-cell constructs.

Assessment of Cell Viability in MAX8-Cell Constructs

Unless noted otherwise, cell viability assays were performed in 384-well plates using the RealTime-Glo™ MT Cell Viability Assay from Promega. For each experiment two stock solutions were prepared. For the first solution, 50 mM HEPES (pH 7.4) buffer was mixed with 0.5 wt % MAX8 (5 mg MAX8 per mL buffer solution for a final 0.25 wt % MAX8 hydrogel construct). The second solution was a cell solution with 11×10⁶ per mL of medulloblastoma cells in serum-free DMEM. The two stock solutions were thoroughly mixed 1:1 to create a cell/gel mixture and 4 μL of the mixture was added per well to a white 384 well assay plate (Corning) containing 41 μL culture medium per well. The cells were allowed to equilibrate for 24 hours before any luminescence was determined. 10× RealTime-Glo was added in 5 μL of media to each well already containing 45 μL to a final concentration of 1×. For cell viability assays in 96-well plates, the following volumes were used. 74 μL of DMEM cell media containing serum was added to each well, 16 μL of the previously mentioned 0.25 wt % MAX8 cell/gel construct was then added to each well. 10 μL of 10× RealTime-Glo was added to the wells for a final concentration of 1×. The plate was incubated for 60 minutes at 37 C and the luminescence was measured using an Envision Multilevel Reader (Perkin Elmer).

For CellTiter-Glo and CellTiter-Glo 3D assays hydrogel cell constructs were prepared as described above for the RealTime-Glo assay in 384 and 96 well formats. To measure cell viability in 384 well plates 45 μL of either CellTiter-Glo or CellTiter-Glo 3D was added to the well, to measure cell viability in 96 90 μL of either CellTiter-Glo or CellTiter-Glo 3D was added to the well. The plate was incubated for 30 minutes at 25 C and the luminescence was measured using an Envision Multilevel Reader (Perkin Elmer).

Treatment of MAX8-Encapsulated Medulloblastoma Cells with Chemotherapeutics

Vismodegib was added into the surrounding tissue culture medium of MAX8 hydrogel-cell constructs after 24 hours of cell seeding at the indicated concentrations. To limit the exposure of cultured cells to dimethyl sulfoxide (DMSO) from stock solutions of test compounds, 3.5 μL of stock solution was diluted in 1 mL of culture medium and then 20 μL of this solution was added to each well using the Janus workstation (PerkinElmer). Following 48 hours of cell culture the cell viability was measured as previously described using RealTime-Glo with a final concentration of 1×.

Realtime PCR

Total RNA was extracted from 3D cell constructs according to standard procedures. Briefly, cell/gel constructs were created in 24-well plates using 2 mL of serum containing DMEM and 100 μL of the cell/gel construct prepared as described above. After 72 hours of culture the cell culture medium was removed and 0.33 ml of TRIzol (Thermo Fischer) was added to each well. 3 wells with identical culture conditions were then combined for a final volume of 1 mL TRIzol and vigorously pipetted. The TRIzol RNA extraction protocol was then followed according to manufacturer's instructions.

First-strand cDNA was synthesized from 1 μg of RNA using the iScript cDNA Synthesis kit (Bio-Rad, Hercules, Calif.). Quantitative PCR analysis was performed with a SYBR Green PCR master mix using an ABI Prism 7900 Sequence Detection System (both from Applied Biosystems, Foster City, Calif.) and normalized to β2-microglobulin. The primer sequences used for qPCR analyses were nestin, forward 5′-GAGAACTCCCGGCTGCAAAC-3′ and reverse 5′-CTTGGGGTCCTGAAAGCTGAG-3′; gli3, forward 5′-CGAACAGATGTGAGCGAGAA-3′ and reverse 5′TTGATCAATGAGGCCCTCTC-3′; and snail1, forward 5′-GAGCCCAGGCACTATTTCA-3′ and reverse 5′-TGGGAGACACATCGGTCAGA- 3′.

QC Plates

Owing to its unique shear force properties, MAX8-medulloblastoma cell constructs were dispensed at room temperature into 384-well plates (2,000 cells in 4 μL of hydrogel) (Brandtech) with 50 μL of culture medium (DMEM with serum) using a BioTek microplate dispenser. The cells were allowed to equilibrate for 24 hours at 37° C. in the presence of 5% CO₂ and test compounds were added using the Janus work station. Stock drugs stored in DMSO were added to media intermediate plates using the 384 well pin-tool, 40 nL of drug into 20 μL of cell culture media. 10 μL of the intermediate drug media was then added to the QC plates, and following 48 h of cell culture the cell viability was measured using RealTime-Glo and the previously described method.

Statistical Analysis

Data are presented as mean±SD unless otherwise indicated. Differences between means of two groups were analyzed with a two-tailed unpaired Student's t-test and when applicable, P values were determined with P<0.5 denoting statistical significance.

Results

MAX8 β-hairpin Hydrogel

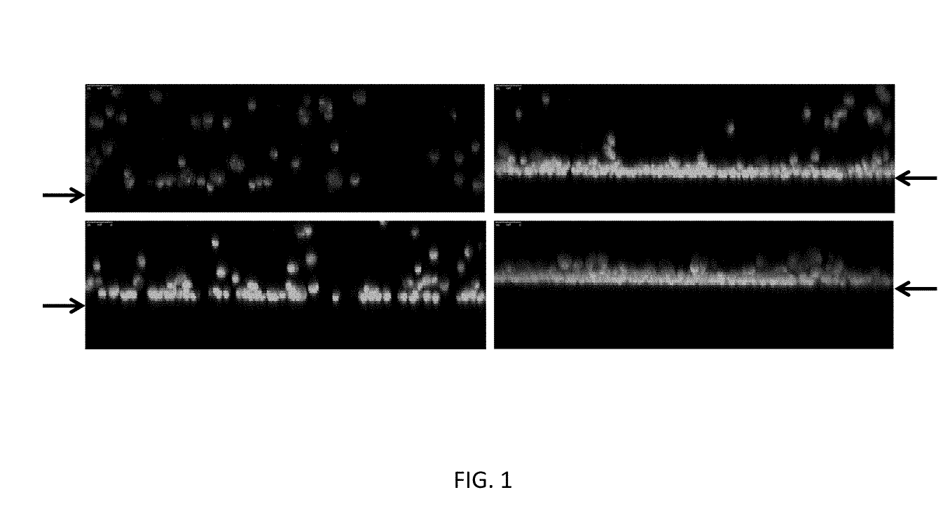

MAX8, a derivative of the originally described MAX1 β-hairpin hydrogel with a single amino acid substitution, is an amphiphilic peptide with the sequence VKVKVKVK-(V^{D}PPT)-KVEVKVKV-NH₂. Gelation can be triggered at room temperature at physiological salt concentration and pH leading to charge screening. This causes the peptide to fold into a β-hairpin and the folded peptides then associate into fibrils forming a network through physical bonds. Gelation triggered by physiological conditions allows for easy culture setup without requiring the addition of harmful chemicals or organic reagents. Unlike commonly used 3D matrices such as collagen and MATRIGEL® matrix (Corning Life Sciences, Tewksbury Mass.), MAX8 gelates within a minute, leading to a homogenous distribution of cells throughout the cell-gel construct (FIG. 1 top left). The stiffness of the hydrogel is around 1000 Pa, and can be controlled by changing the weight percent of the peptide. One critical property of MAX8 for 3D HTS is shear thinning, which allows for hydrogel injection while protecting cells from shear forces, thus making it suitable for automatic handling with standard HTS equipment.

Establishment of 3D MAX8-Cell Constructs

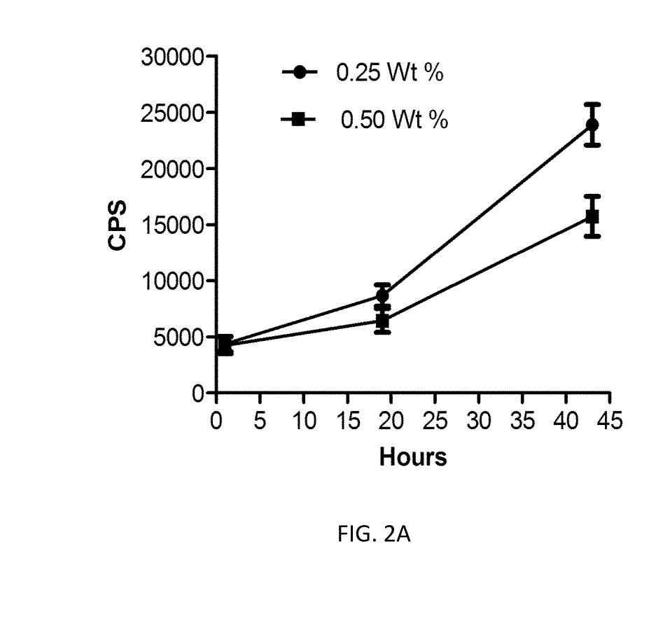

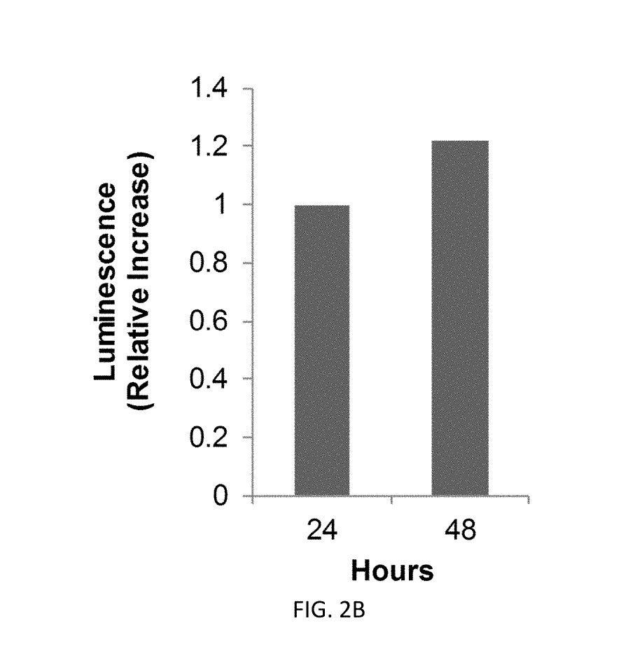

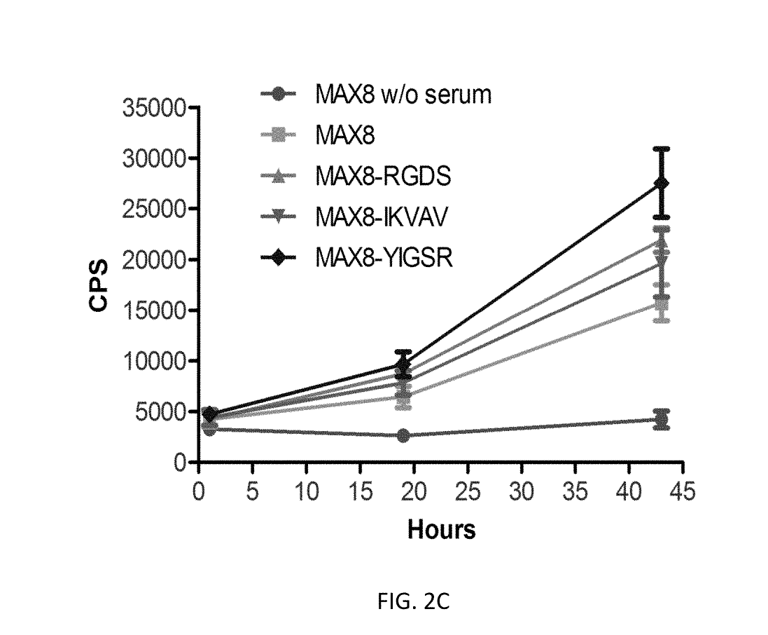

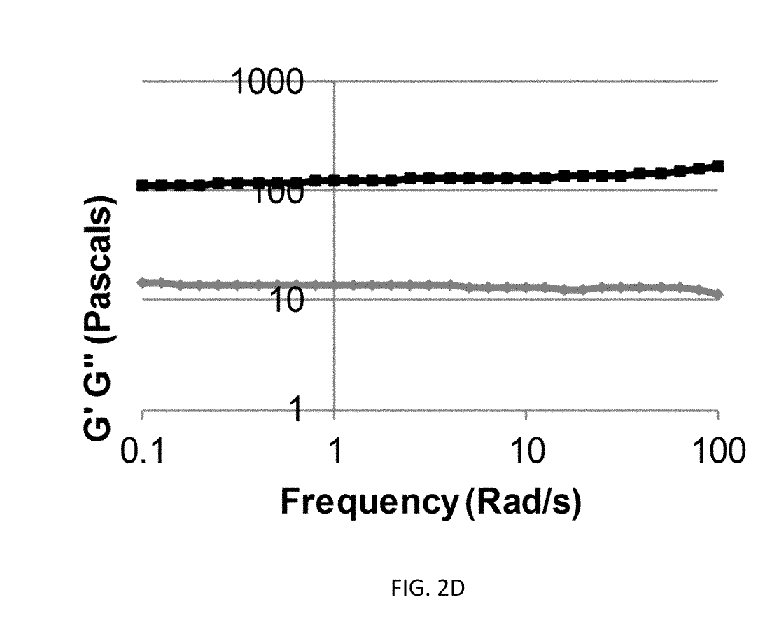

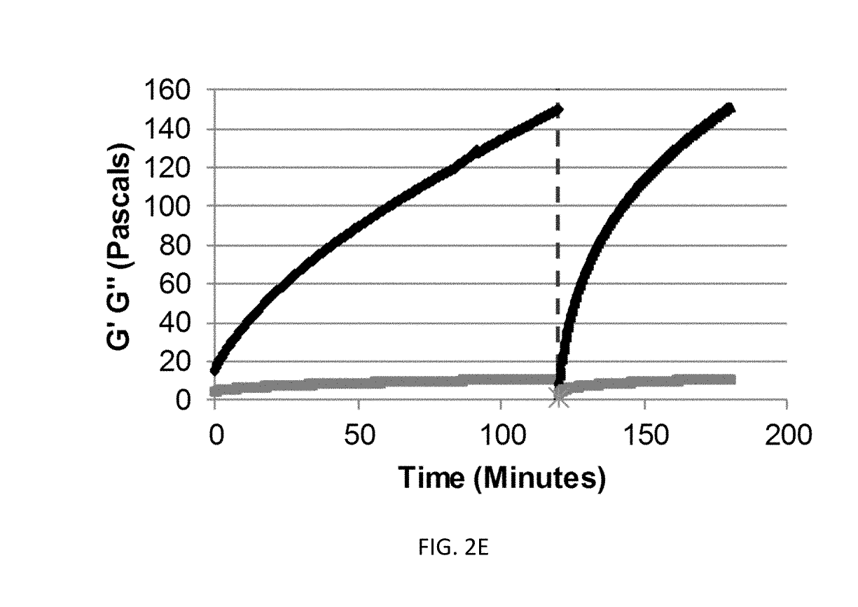

Peptide hydrogels are ideal materials to use as 3D cell culture scaffolds because of the similarities in material properties and properties of biological extracellular matrix. Even in the absence of adhesive ligands, native MAX8 is compatible with cells of various origin, including human medulloblastoma cells (FIG. 2A) and primary neuronal cells (FIG. 2B). Both medulloblastoma cells (FIG. 2A) and primary cerebellar granule precursors (CGPs) isolated from wild-type C57BL/6 mice were viable for several days within MAX8-cell constructs as determined by the RealTime-Glo cell viability assay. Encapsulation of medulloblastoma cells in MAX8 revealed that cell proliferation decreased with increasing MAX8 concentrations which is likely due to reduced diffusion of growth factors from the culture medium into the hydrogel at higher peptide concentrations (FIG. 2A). Addition of the RGDS ligand, a peptide sequence found in fibronectin that interacts with integrins and supports cell adhesion, enhanced the proliferation of medulloblastoma cells encapsulated in MAX8-RGDS-cell constructs 1.4-fold over native MAX8 (FIG. 2C). Tagging of MAX8 with IKVAV or YIGSR, both ligands that are normally observed in laminin protein and support neuronal differentiation as well increased the proliferation of medulloblastoma cells when compared to MAX8 (FIG. 2C). This increase in cell proliferation was mostly due to the presence of the adhesive sequences since the addition of the peptide sequence did not substantially change basic material properties MAX8-RGDS with a linear response regime in the frequency sweep (FIG. 2D) and shear thinning properties (FIG. 2E) that were similar to that observed in MAX8. Based on the data obtained the inventors chose MAX8 tagged with the well-characterized RGDS sequence at 0.25 wt % peptide concentration to establish the 3D HTS screening platform.

Comparison of MAX8-Cell Constructs with 2D Cultures

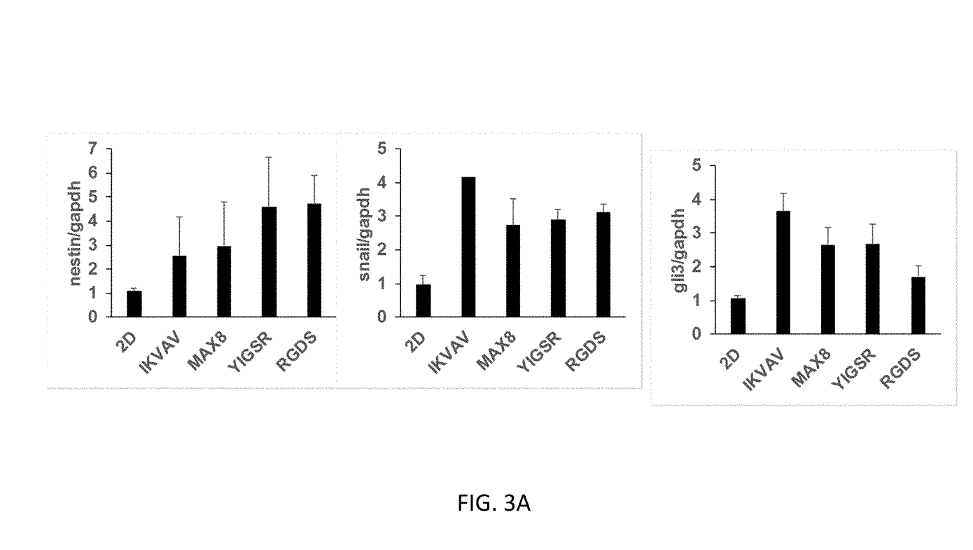

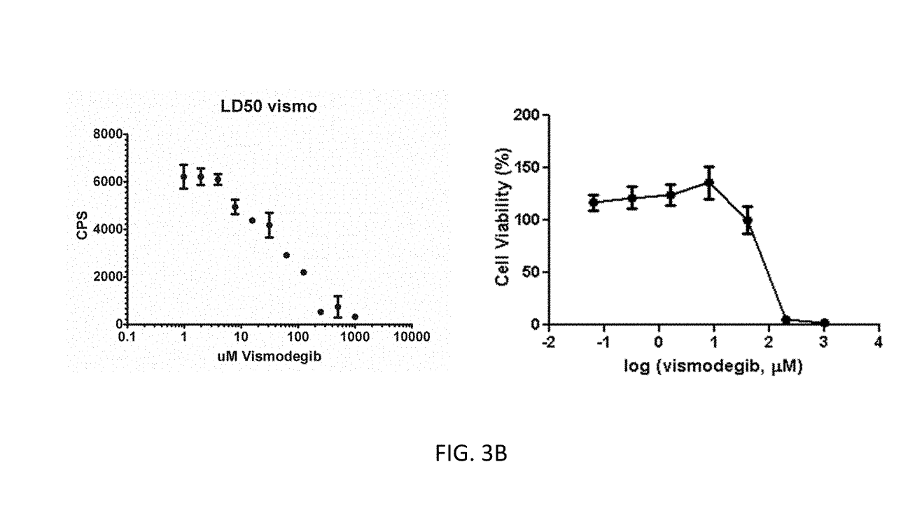

For various cell lines, including medulloblastoma, cells cultured in 2D differ from those in 3D cultures. The inventors compared the expression profiles of various differentiation and stem cell markers in medulloblastoma cells grown in monolayers and with MAX8 and tagged MAX8 cell constructs (FIG. 3A). Interestingly, while some variations were observed between hydrogel constructs with native MAX8 or tagged MAX8, all 3D cultures had higher mRNA levels of nestin, snail and gli3 compared to monolayers, suggesting that the culture of MB cells in 3D supports a cancer stem cell-like phenotype.

The inventors further tested the sensitivity of medulloblastoma cells in MAX-RGDS cell constructs and in monolayers to commonly used chemotherapeutics and vismodegib (FIG. 3B), revealing a shift in dose response curves between monolayers and hydrogel cultures.

Feasibility of MAX8 Constructs as a 3D Scaffold for Automated HTS

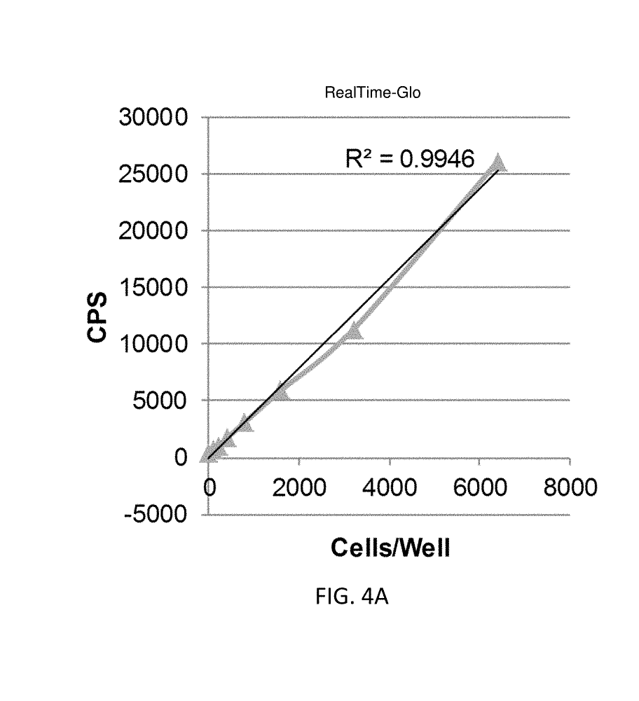

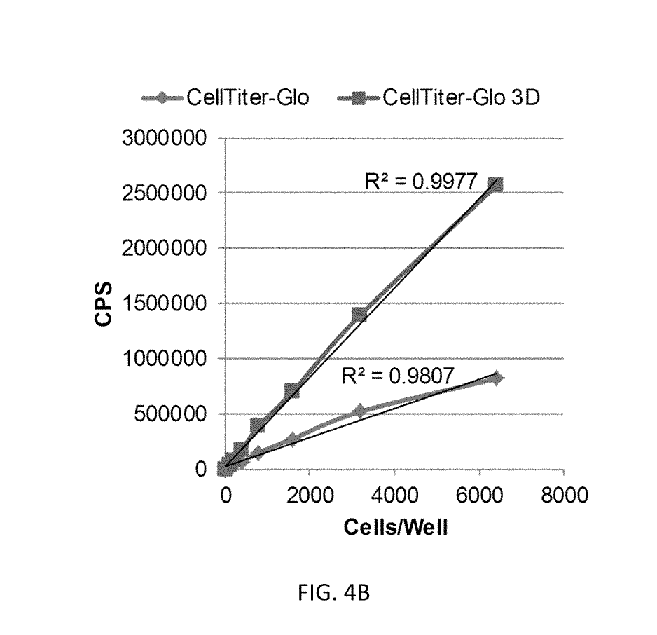

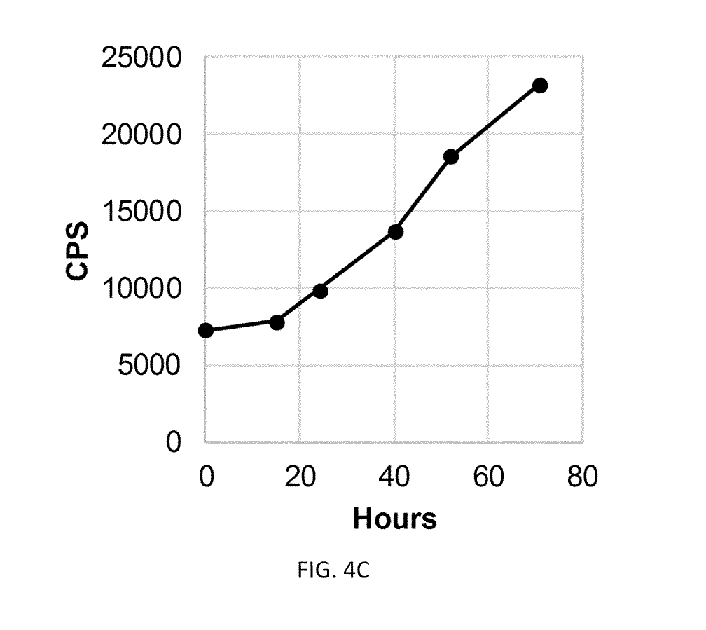

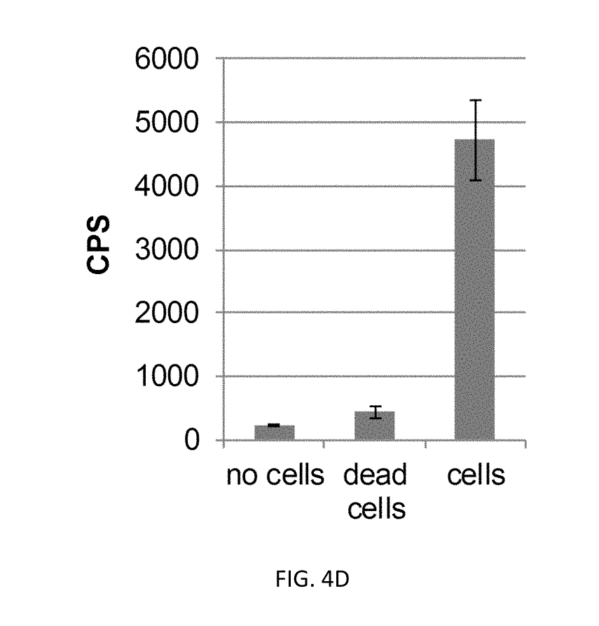

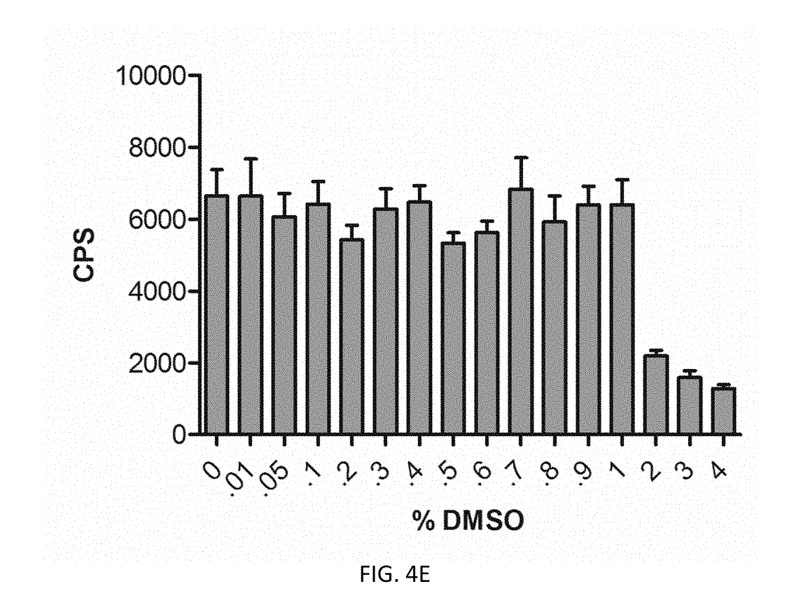

The fast gelation kinetics at room temperature and under physiological are critical properties that make MAX8 suitable for automated handling by standard HTS equipment. The inventors first tested the compatibility of MAX8-RGDS cell constructs with commercially available cell viability assays suitable for HTS. The RealTime-Glo MT Cell Viability Assay is a nonlytic bioluminescent method to measure cell viability in real time and determines the number of viable cells by measuring the reducing potential and thus metabolism of cells. Both, the CellTiter-Glo Luminescent Cell Viability Assay and the CellTiter-Glo 3D Cell Viability Assay determine the number of viable cells based on the quantitation of ATP present. However, the CellTiter-Glo 3D assay is formulated with more robust lytic capacity for use in 3D cell culture. All three assays showed a strong correlation between signal and number of viable cells (FIGS. 4A, B) making them well suited for cytotoxicity studies. While the overall luminescence signals obtained with both the CellTiter-Glo and the CellTiter-Glo 3D assays (FIG. 4B) were about 100-fold higher than the signal obtained from the RealTime-Glo assay (FIG. 4A), the encapsulation of medulloblastoma cells into MAX8-RGDS hydrogel allowed for detection of a robust signal of proliferating cells (FIG. 4C) with a calculated Z-factor of 0.576 (FIG. 4D). Due to its capability for longitudinal tracking of cell growth in a single sample the inventors decided to proceed with the RealTime-Glo assay.

In preparation for HTS screening, the inventors tested the DMSO tolerance of medulloblastoma cells in hydrogel-cell constructs and determined the overall quality of the 3D HTS setup. Medulloblastoma cells were viable at DMSO concentrations of up to 1% (FIG. 4D). Thus, the hydrogel environment does not adversely affect the sensitivity of medulloblastoma cells to DMSO and, in addition, 0.05% DMSO introduced by 50 nL pintool delivery of test compounds in a HTS screen will not be of concern. Furthermore, dispensation of MAX8-RGDS hydrogel-cell mixtures into 384-well cells using a Janis microplate dispenser proofed to be reproducible and reliable (FIG. 4E).

Discussion 3D HTS is a rapidly expanding section of the drug discovery process that is predicated on the idea that using a disease model which is a more accurate recapitulation of the in vivo environment will provide more clinically actionable results. However, until recently most 3D culture technologies had limited automation possibilities, scalability and reproducibility. Here the inventors have shown that MAX8 β-hairpin hydrogel with its well-defined material characteristics, unique solution assembly and flow shear properties can overcome these limitations and provide a suitable 3D cell culture scaffold for HTS. Medulloblastoma cells incorporated into MAX8 and automatically dispensed into 384-well plates showed robust cell proliferation with the proliferation rate depending on peptide concentration. The addition of ligand peptides found in extracellular matrix such as RGDS, IKVAV and YIGDR increased medulloblastoma cell proliferation within 3D hydrogel-cell constructs. Differences in cell phenotype were confirmed by determining the mRNA levels of stem cell and differentiation markers that revealed that medulloblastoma cells grown in 3D hydrogels express more stem cell markers than cells grown in monolayers. Primary cultures of mouse cerebellar granule cells proliferated at a slower rate, but even native MAX8 was compatible with primary cells. Using the RealTime-Glo™ MT Cell Viability Assay, the inventors standardized a sensitive HTS-compatible cell viability assay with a robust Z-score. As MAX8 has been shown to be compatible with a variety of cell lines and primary cells, and can be incorporated into standard HTS equipment, the inventors expect MAX8 and its derivatives to have broad applicability as a versatile cell culture scaffold for 3D HTS.

Although the invention is illustrated and described herein with reference to specific embodiments, the invention is not intended to be limited to the details shown. Rather, various modifications may be made in the details within the scope and range of equivalents of the claims without departing from the invention.

1. An assay mixture comprising a hydrogel of a shear-thinning hairpin peptide, a plurality of cells, and one or more predetermined compounds being investigated for ability to affect the growth, viability, reproduction characteristics, or activity of the cells.

2. The assay mixture of claim 1, wherein the hairpin peptide is MAX8 or a derivative thereof.

3. The assay mixture of claim 1, wherein the stiffness of the assay mixture is within 50% above or below the stiffness of an in vivo tissue in which the growth, viability, reproduction characteristics, or activity of like cells is sought to be affected.

4. The assay mixture of claim 1, wherein the cells are distributed randomly or evenly throughout the assay mixture.

5. The assay mixture of claim 1, wherein each of the one or more predetermined compounds is distributed randomly or evenly throughout the assay mixture.

6. The assay mixture of claim 1, wherein the cells are distributed in a layer of the assay mixture while another layer of the assay mixture contains no cells.

7. The assay mixture of claim 1, wherein at least one of the one or more predetermined compounds is distributed in a layer of the assay mixture while another layer of the assay mixture contains none of said predetermined compounds.

8. The assay mixture of claim 1, wherein the cells are eukaryotic cells.

9. The assay mixture of claim 1, wherein the cells are cancer cells.

10. The assay mixture of claim 1, wherein the cells are medulloblastoma cells.

11. The assay mixture of claim 1, wherein the cells are bacterial cells.

12. The assay mixture of claim 1, wherein the cells are fungal cells or spores thereof.

13. The assay mixture of claim 1, wherein the cells are plant cells.

14. The assay mixture of-any one of claim 1, wherein the cells are medulloblastoma cells and are mixed homogeneously throughout the entire hydrogel.

15. The assay mixture of claim 1, wherein the cells are medulloblastoma cells present as superstructures (spheroids).

16. The assay mixture of claim 1, comprising a bottom layer of hydrogel without cells, a middle layer of hydrogel containing fibroblast cells (3T3), a top layer of hydrogel without cells, and a cell monolayer of keratinocytes or human embryonic kidney cells cultured on top of the top layer of hydrogel.

17. The assay mixture of claim 1, having at least two layers of differing hairpin weight percent content.

18. The assay mixture of claim 1, wherein a cell ligand is covalently bonded to the shear-thinning hairpin peptide.

19. The assay mixture of claim 1, wherein multiple cell types are co-cultured therein.

20. A high throughput screening device comprising a plurality of sample wells adapted for high throughput screening, wherein each well contains an assay mixture according to claim 1, and wherein the one or more compounds and/or the amounts thereof may be the same or different from well to well, provided that some but not all of the wells may optionally be control wells containing no compounds to be investigated.

21. A method of using the device of claim 20 for high throughput screening of compounds for ability to affect the growth, viability, reproduction characteristics, or activity of cells, comprising
a) depositing in each of the wells a hairpin hydrogel comprising the cells;
b) depositing in at least some of the wells one or more of the compounds, either along with the hairpin hydrogel or separately; and
c) measuring the growth, viability, reproduction characteristics, or activity of the cells in each of the plurality of wells.
